(19) United States
(12) Patent Application Publication
(10) Pub. No.: US 2012/0209203 A1
(43) Pub. Date: Aug. 16, 2012

(54) MEDICAL DEVICE FOR APPLYING CATHETERS, PARTICULARLY FOR THORACENTESIS PROCEDURES

(76) Inventors: Lucio Gibertoni, Mirandola (IT); Andrea Gibertoni, San Possidonio (IT)

(21) Appl. No.: 13/502,155

(22) PCT Filed: Oct. 4, 2010
(86) PCT No.: PCT/EP2010/064761

§ 371 Date: Apr. 16, 2012

(30) Foreign Application Priority Data
Oct. 15, 2009 (IT) MI2009A001772

(51) Int. Cl. A61M 27/00; A61B 17/34

(52) U.S. Cl. 604/164.11; 604/317

(57) ABSTRACT

A medical device for applying catheters, particularly for thoracentesis procedures, comprising a hollow supporting element which can be gripped ergonomically and accommodates a selector which is adapted to connect at least three openings defined in the supporting element, the first opening being functionally associated with a cannula adapted to be inserted in the body of a patient, the second opening and the third opening being able to be functionally associated respectively with two closure elements or with at least one of a syringe and a collection bag, a sharp-tipped needle being slidingly insertable in the cannula and having such a length as to pass through at least all of the cannula and protrude from it, the device further comprising a fourth opening defined in the supporting element on the side opposite the first opening for extraction or insertion of the sharp-tipped needle respectively from and into the cannula.

### CROSS-REFERENCE TO RELATED APPLICATION

This application hereby claims priority to Italian Patent Application No. MI2009A001772, filed on 15 Oct. 2009, the entire content of which is hereby incorporated by reference in its entirety.

### TECHNICAL FIELD

The present invention relates to a medical device for applying catheters, particularly for thoracentesis procedures.

### BRIEF DESCRIPTION OF RELATED ART

Thoracentesis is a procedure adapted to remove fluids, both in the liquid state and in the gaseous state, from the pleural space by means of a large needle or a small cannula made of plastics, termed more commonly catheter, which can be inserted in the needle or more commonly covers such needle so as to leave it connected to the pleural space once the needle has been removed.

More precisely, the procedure entails retracting the metal tip of the needle from the catheter as soon as the tip of the catheter makes contact with the liquid to be drained. Subsequently, one advances a little further inside the pleural space and the entire metallic portion is retracted from the catheter, which is pushed completely inside the pleural space so as to allow the complete evacuation of the liquid without the risk of piercing the lung.

The catheter is inserted in the body of the patient by performing a small cut, measuring by way of indication 2 millimeters, in the skin of the patient with the tip of a scalpel in order to prevent the catheter itself, which covers the metallic needle, from gathering on the needle while it attempts to penetrate the skin.

In latest-generation medical devices of the known type adapted to manage and apply catheters, inside the sharp-tipped needle, accommodated in the cannula made of plastics, there is a round-tipped element, which is spring-loaded and protrudes beyond the sharp tip of the needle and retracts into the needle while the thoracic wall is pierced. As soon as the wall is passed, i.e., as soon as there is no more resistance, by action of the spring the round-tipped element protrudes again past the sharp tip of the needle.

This solution is advantageous because as the liquid is evacuated, the lung, by expanding, comes into contact with the tip of the round-tipped element and not with the tip of the sharp-tipped needle, thus avoiding accidental puncture of said lung.

Generally, the system just described is supported by a hollow supporting element with three openings, two of which are mutually opposite, to which the plastic cannula, a syringe for drugs and a bag for collecting the liquid are connected respectively.

More precisely, inside the supporting element there is a manually actuated selector by means of which it is possible to connect the plastic cannula, associated with the first opening of the supporting element, to one or both of the remaining openings.

Practically, when the catheter is inserted in the pleural space, the opening that lies opposite the one associated with the catheter is free so as to allow the extraction of the sharp-tipped needle once the thoracic wall of the patient has been pierced and the catheter has been oriented correctly inside the pleural space.

In fact, during the step of applying the catheter, it is possible to orient the latter inside the pleural space by directly moving the sharp-tipped needle that is present therein.

After the extraction of the sharp-tipped needle, it is possible to connect the syringe to the supporting element, if necessary.

Differently, the collection bag is connected immediately to the supporting element, at the opening that is not opposite the one associated with the catheter, so as to collect immediately the liquid to be drained that exits from the patient also during the steps for applying the catheter.

These know types of medical device are not devoid of drawbacks, which include the fact that once the catheter has been applied to the patient, when the catheter is no longer able to aspirate the liquid to be drained because of the lowering of the latter in the pleural space, the catheter must be removed and replaced with another one, inserted with a new orientation, so as to draw the remaining portion of the liquid to be drained.

Another drawback consists in that during the process for aspirating the fluid, by means of a syringe, currently commercially available products force the operator to switch the flow diverter valve at every single stroke of the piston of the syringe. In other words, during suction (traction of the piston in the extended position) the selector must be in a specific position for connecting the syringe proper to the thorax of the patient; during the return step of the piston to the initial position, the selector must be switched to a different position so as to connect the syringe to the collection bag.

Selector switching must be performed with each suction and discharge, until the end of the drainage operation. Another drawback of existing products is the presence of the one-way valve in the grip of the sharp-tipped needle. This is to allow unidirectional suction of a small quantity of liquid and to verify the positioning of the needle inside the bag of fluid to be drained, before extracting such bag.

This leads to an increase in the dimensions of such grip, with greater handling difficulty. Moreover, exploratory suction is performed with a syringe different from the one that will be used subsequently for the drainage, i.e., a smaller one, with the consequent need to supply it with the product and an increase in the degree of complexity of the kit and its cost.

Another drawback of medical devices of the known type consists in that the medical staff assigned to apply the catheter does not have any feedback on the position of the catheter inside the body when it is applied, i.e., whether the catheter has already entered the pleural space or is still passing through the tissues of the ribcage.

In other words, the medical staff assigned to apply the catheter, with medical devices of the known type, does not have any feedback on the position of the round-tipped element with respect to the sharp-tipped needle, i.e. on whether the former is protruding from the latter or is retracted completely or partially inside the latter.

### BRIEF SUMMARY

The aim of the present invention is to provide a medical device for applying catheters, particularly for thoracentesis procedures, which allows repositioning of the catheter inside the pleural space after the application steps without necessarily extracting and reapplying the catheter as in the background art.

Within this aim, a medical device is provided for applying catheters that gives constant feedback on the position of the catheter inside the body during its application.

Additionally, a medical device is provided for applying catheters that is structurally simple and whose manufacturing costs are competitive with respect to those of the background art.

More specifically, a medical device is provided for applying catheters, particularly for thoracentesis procedures, comprising a hollow supporting element which can be gripped ergonomically and accommodates a selector which is operated manually and is adapted to connect at least three openings defined in said supporting element, the first one of said openings being functionally associated with a cannula, which is adapted to be inserted in the body of a patient for the drainage of biological liquid or for the injection of drugs, the second opening and the third opening being able to be functionally associated respectively with two closure elements or with at least one of a syringe and a collection bag, a sharp-tipped needle being able to be inserted slidingly in said cannula and having such a length as to pass through at least said entire cannula and protrude from said cannula to pierce the biological tissue that lies in the vicinity of said biological liquid, characterized in that it comprises a fourth opening defined in said supporting element on the side opposite said first opening for extraction or insertion of said sharp-tipped needle, respectively, from and into said cannula.

### BRIEF DESCRIPTION OF THE DRAWINGS

Further characteristics and advantages of the present invention will become better apparent from the description of two preferred but not exclusive embodiments of a medical device for applying catheters, according to the invention, illustrated by way of non-limiting example in the accompanying drawings, wherein:

FIG. 1 is a partially sectional side elevation view of the first embodiment of the medical device for applying catheters, particularly for thoracentesis procedures, according to the invention;

FIG. 2 is a plan view of the medical device for applying catheters according to the invention;

FIG. 3 is a sectional view, taken along the line III-III, of the medical device illustrated in FIG. 2;

FIG. 4 is a partially sectional side elevation view of the medical device illustrated in FIG. 1 during the step of insertion of the catheter through the tissues that lie in the vicinity of the biological liquid to be drained;

FIG. 5 is a longitudinal sectional view of the medical device illustrated in FIG. 4;

FIG. 6 is a partially sectional side elevation view of the medical device illustrated in FIG. 1 at the end of step of inserting the catheter;

FIG. 7 is a longitudinal sectional view of the medical device illustrated in FIG. 6;

FIGS. 8 and 9 are two partially sectional side elevation views of the second embodiment of the medical device for applying catheters, particularly for thoracentesis procedures, according to the invention;

FIG. 10 is a perspective view of the medical device illustrated in FIGS. 8 and 9 during the step of insertion of the catheter through the tissues that lie in the vicinity of the biological liquid to be drained;

FIG. 11 is a perspective view of the medical device illustrated in FIGS. 8 and 9 during the step of draining the biological liquid to be drained;

FIGS. 12 and 13 are two perspective views of the medical device illustrated in FIGS. 8 and 9 during the step of repositioning the catheter;

FIGS. 14 and 15 are two perspective views of the medical device illustrated in FIGS. 8 and 9 during the step of draining the biological liquid to be drained.

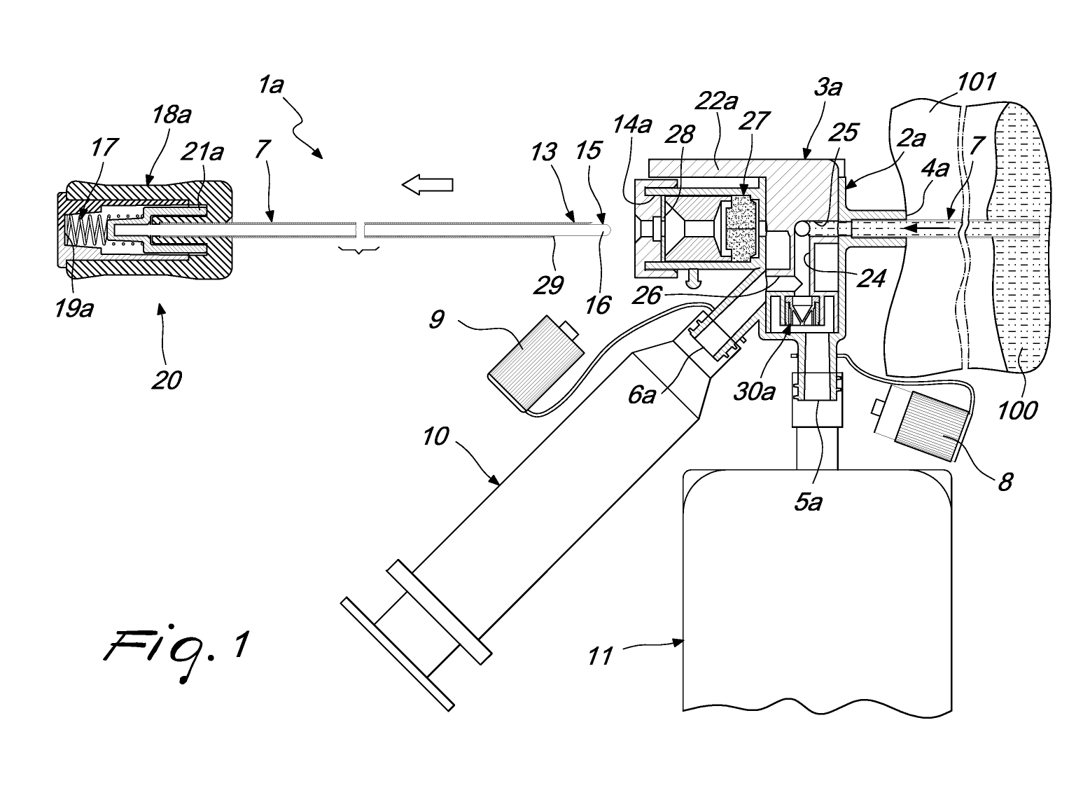

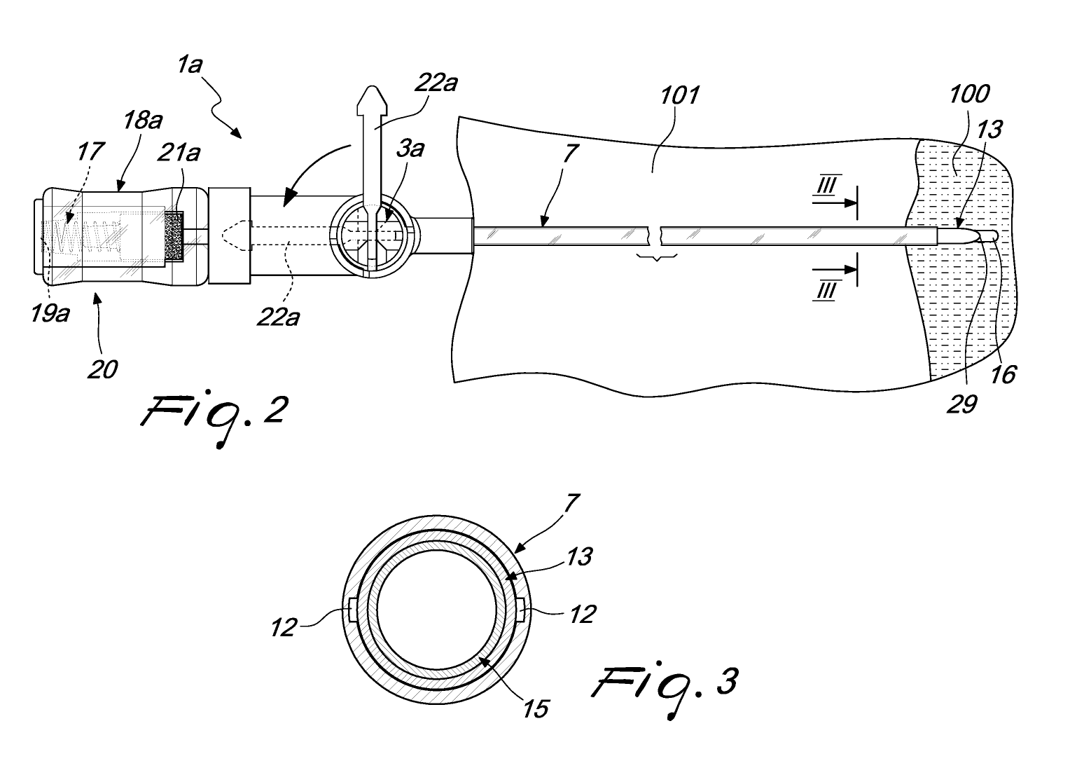

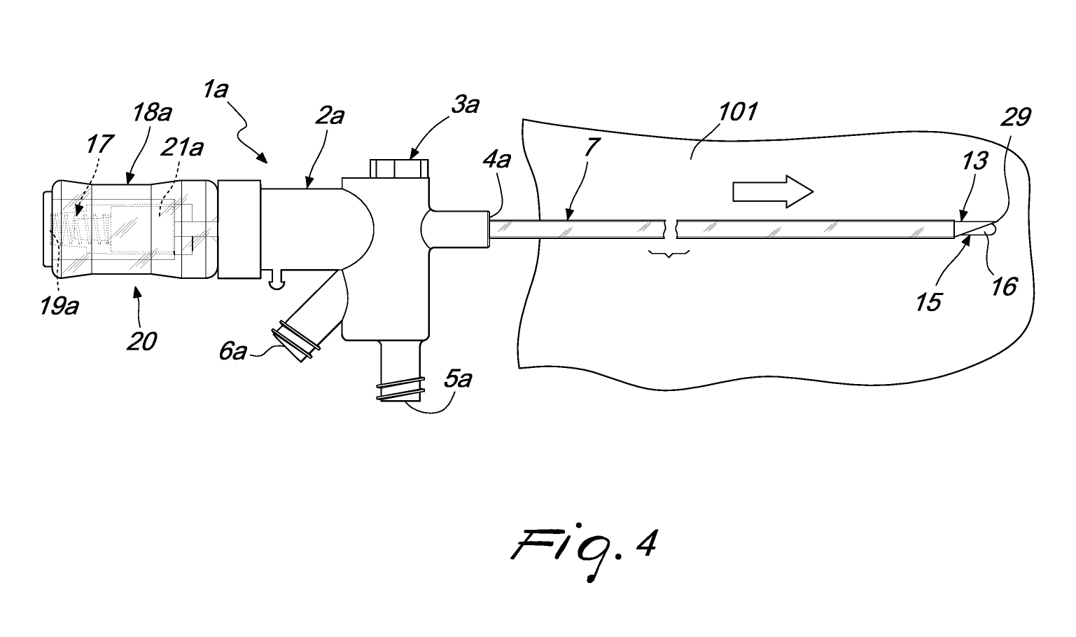

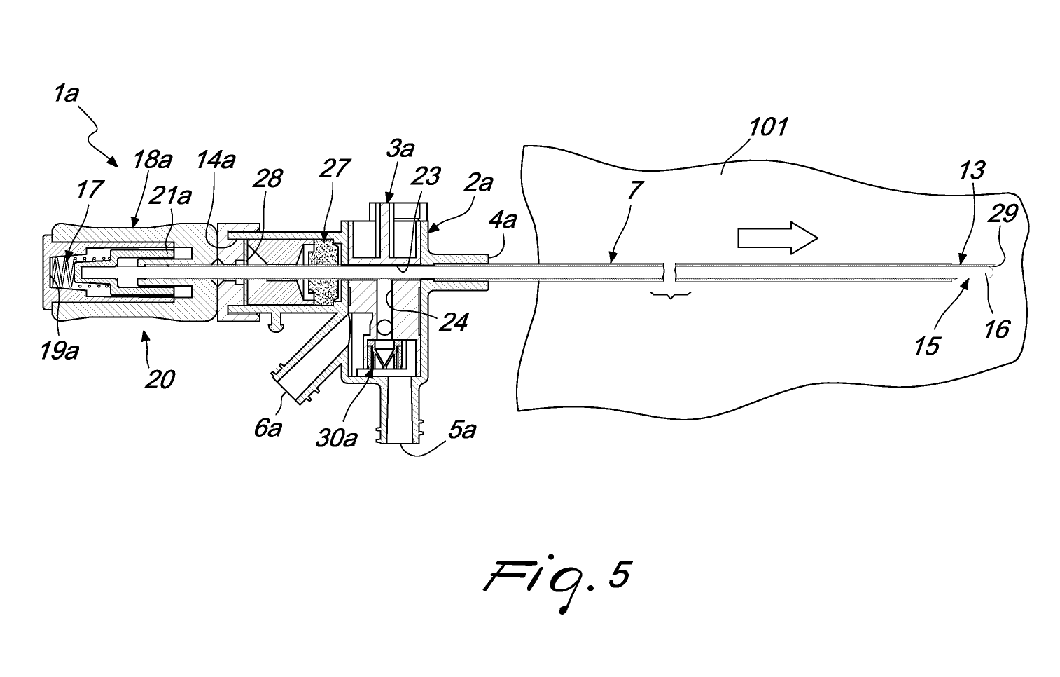

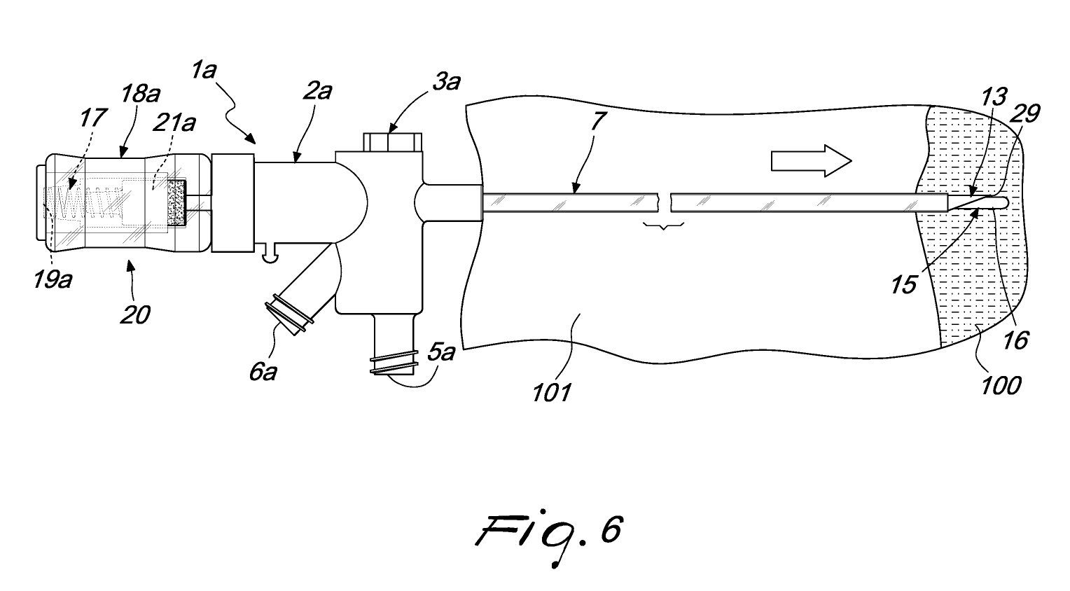

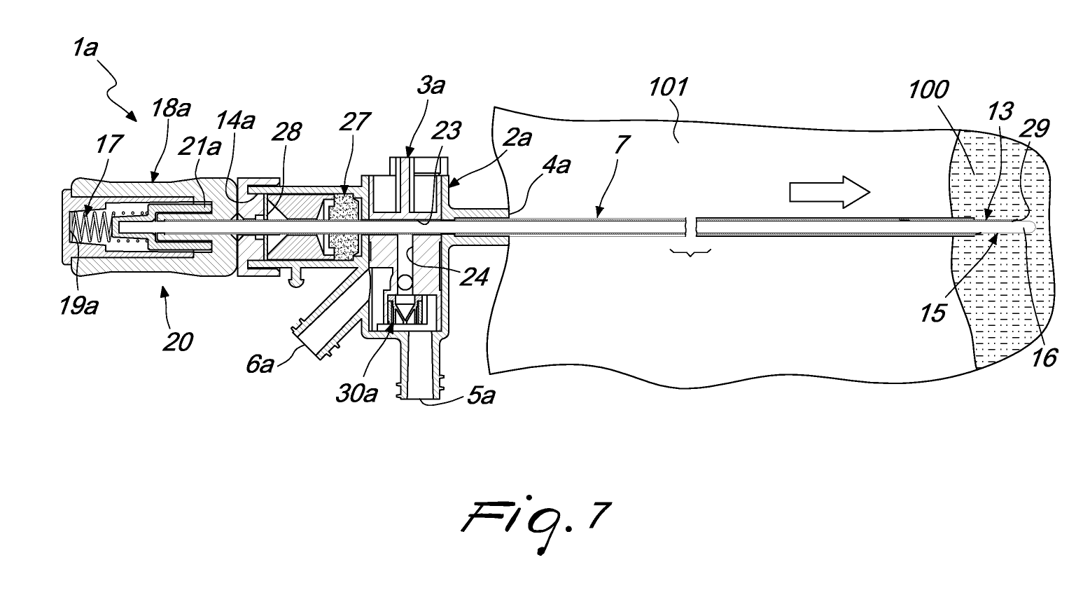

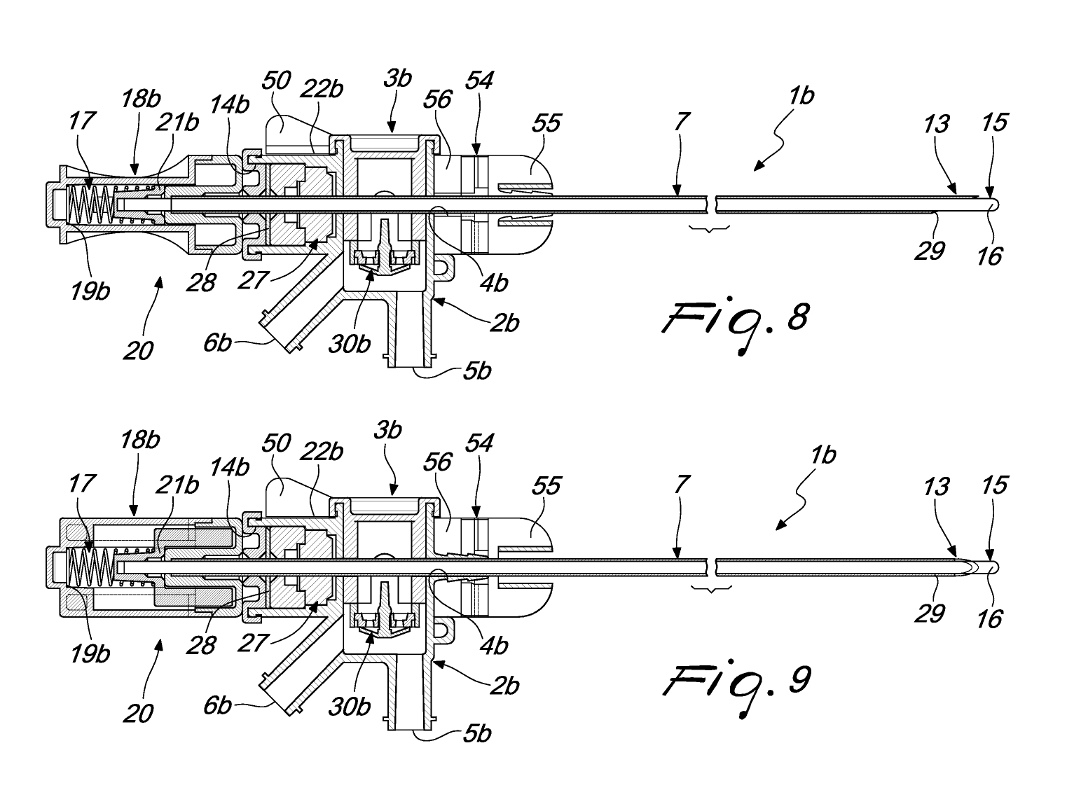

### DETAILED DESCRIPTION

With reference to the figures, the medical device for applying catheters, particularly for thoracentesis procedures, is generally designated in the two proposed embodiments by the reference numerals la and lb.

Hereinafter, the elements that are common to both embodiments will be designated by the same numbering and the elements that are present in both embodiments but have geometric variations between one embodiment and the other will be numbered by adding the letter “a” as suffix for the first embodiment and the letter “b” as suffix for the second embodiment.

The medical device 1a or 1b comprises a hollow supporting element 2a or 2b, which can be gripped ergonomically and accommodates a selector 3a or 3b, which is operated manually and is adapted to connect at least three openings 4a, 5a and 6a or 4b, 5b and 6b defined in the supporting element 2a or 2b.

More precisely, in both of the proposed embodiments, the first opening 4a or 4b is functionally associated with a cannula 7, which is adapted to be inserted in the body of a patient to drain biological liquid 100 or to inject drugs, and the second opening 5a or 5b and the third opening 6a or 6b, which have standard connectors for example of the female LUER-LOCK type, can instead be functionally associated respectively with two closure elements 8 and 9 or with at least one of a syringe 10 and a collection bag 11, which can have a reservoir made of silicone for the forced aspiration of the biological liquid 100 to be drained.

Advantageously, it is possible to insert slidingly in the cannula 7, which can comprise at least one groove 12 that is provided on its inner surface and runs longitudinally with respect to the cannula 7 for the constant connection of the first opening 4a or 4b to the biological liquid 100 to be drained, a sharp-tipped needle 13 whose length is such as to pass through at least the entire cannula 7 and protrude from it so as to pierce the biological tissue 101 that lies in the vicinity of the biological liquid 100 to be drained. According to the invention, each one of the supporting elements 2a and 2b forms, on the side opposite the first opening 4a or 4b, a fourth opening 14a or 14b for extraction or insertion of the sharp-tipped needle 13, respectively, from and into the cannula 7 without involving the openings 5a or 5b and 6a or 6b.

In other words, in this manner an opening is provided which is dedicated only to the operations of extraction and insertion of the sharp-tipped needle 13, which can be moved without necessarily detaching any syringes 10 and collection bags 11 connected to the supporting element 2a or 2b.

More precisely, a round-tipped element 15 is inserted slidingly in the sharp-tipped needle 13 so that its round tip 16 normally protrudes from the sharp-tipped needle 13 on the side opposite the supporting element 2a and 2b. Such round tip 16 is retractable into the sharp-tipped needle 13 in opposition to the action of elastic means 17 associated with the round-tipped element 15 and following the impact of the round tip 16 against a body that has such a consistency as to make it retract, such as, for example, the biological tissue 101 that lies in the vicinity of the pleural space.

As regards the elastic means 17, they comprise a helical spring, which is accommodated in a container body 18a or 18b, which is jointly connected to the sharp-tipped needle 13 and can engage the supporting element 2 at the fourth opening 14a or 14b.

More precisely, the helical spring of the elastic means 17 is interposed between one end of the round-tipped element 15 and an internal abutment surface 19a or 19b of the container body 18a or 18b.

Advantageously, for having a visual feedback of the retraction of the round tip 16 into the sharp-tipped needle 13 during use of the medical device 1, visualization means 20 are provided which comprise a slider 21a or 21b, associated with the round-tipped element 15 on the side opposite the round tip 16.

The slider 21a or 21b is accommodated in the container body 18a or 18b and can move from a visible position, in which the round tip 16 protrudes at least partially from the sharp-tipped needle 13, to a non-visible position, in which the round tip 16 is completely retracted into the sharp-tipped needle 13 as a consequence of the translation of the round tip 16.

Conveniently, this visibility is made possible by the fact that the container body 18a or 18b is at least partially or totally transparent for viewing the slider 21a or 21b in its visible position.

Moreover, the container body 18a or 18b has such a shape that it can be gripped easily by medical staff so as to facilitate the operations of insertion and extraction of the sharp-tipped needle 13 in and from the cannula 7.

More precisely, both the supporting element 2a and 2b and the container body 18a and 18b in the first embodiment and in the second embodiment have reduced dimensions for increasing the comfort of the patient during prolonged uses, i.e., in cases in which the medical device 1b has to remain in place for several days. Obviously, the reduced dimensions must not be detrimental to the grip, which must be ergonomic for providing optimum control during the positioning step.

In particular, the supporting element 2b, with respect to the supporting element 2a, eliminates all unnecessary spaces, making it even more compact, and this allows easy use to the physician and, at the same time, a practical fixing in case of long-term applications.

Moreover, the container body 18b has such a geometric shape as to facilitate its gripping by the operating physician. This shape has in fact two mutually opposite concavities, such as to accommodate part of the physician's fingers.

As will be described in more detail hereinafter, during use of the medical device 1a or lb the four openings 4a, 5a, 6a and 14a or 4b, 5b, 6b and 14b can be mutually connected selectively by means of the selector 3a or 3b, which has manually actuated control means, comprising a control lever 22a or 22b that can move from a first functional position to a second functional position.

More precisely, the four openings 4a, 5a, 6a and 14a or 4b, 5b, 6b and 14b are all four mutually connected when the control lever 22a or 22b is arranged in the first functional position.

When the control lever 22a or 22b is arranged in the second functional position, only the first opening 4a or 4b, the second opening 5a or 5b and the third opening 6a or 6b are instead mutually connected.

More particularly, each one of the selectors 3a and 3b comprises a small cylinder inserted in the respective supporting element 2a or 2b substantially at right angles to the insertion axis of the sharp-tipped needle 13 and can rotate about its own axis, thanks to the control lever 22a or 22b, so as to switch it from the first functional position to the second functional position and vice versa.

For further improving the ergonomics of the medical device 1b, the control lever 22b has a flap 50 that protrudes along a direction substantially parallel to the axis of the small cylinder of the selector 3b that is inserted in the supporting element 2b, so as to be gripped easily by the physician.

With reference to the first functional position, the selector 3a or 3b has a first duct 23 that passes entirely through it and connects the first opening 4a or 4b and the fourth opening 14a or 14b.

A second duct 24 branches off from the first duct 23, whose dimensions are such as to allow the passage of the sharp-tipped needle 13, at right angles to said first duct 23 in the direction of the second and third openings 5a or 5b and 6a or 6b to which it is connected. More precisely, with the selector 3a or 3b arranged in the first functional condition, the openings 5a or 5b and 6a or 6b are connected to each other and the second duct 24 is connected only to the second opening 5a or 5b.

With reference to the second functional position, the selector 3a or 3b has a third duct 25, which is adapted to connect the first opening 4a or 4b to the second duct 24, which in turn is connected to the second opening 5a or 5b.

In this second functional position, the openings 5a or 5b and 6a or 6b are not connected directly to each other but are both connected to the third duct 25, respectively, through the second duct 24, which is connected directly to the second opening 5a or 5b and to the third opening 6a or 6b by means of a fourth duct 26, which is connected to the second duct 24.

In this manner, the fourth opening 14a or 14b is isolated from the other three openings 4a, 5a and 6a or 4b, 5b and 6b.

Advantageously, in both of the supporting elements 2a and 2b there is a first valve 27 of the one-way normally-closed type, which is interposed between the respective selector 3a or 3b and the respective fourth opening 14a or 14b and has a membrane-type sealing gasket 28 which can engage by abutment against the respective container body 18a or 18b if present.

More precisely, this first valve 27 has a central slit which is elastically deformable for the passage of the sharp-tipped needle 13 in both directions so as to make it possible to insert it and extract it from the cannula 7.

In order to avoid leaks due to the difference in diameter and shape between the sharp-tipped needle 13, its sharp part 29 and the round tip 16 of the round-tipped element 15, the first valve 27 and the membrane-type sealing gasket 28 are arranged at a distance from each other that is greater than the sum of the length of the sharp part 29 of the sharp-tipped needle 13 and the stroke of the round tip 16 of the round-tipped element 15.

With this solution, it is possible to prevent unwanted leaks of the biological liquid 100 to be drained during the operations for inserting and extracting the sharp-tipped needle 13 in and from the cannula 7.

In both of the supporting elements 2a and 2b, a respective second valve 30a or 30b of the one-way type is accommodated and interposed between the respective selector 3a or 3b and the second opening 5a or 5b.

More precisely, in the first embodiment and in the second embodiment the second valve 30a and 30b comprises an umbrella valve that has a reduced vertical size, promoting the compactness of the supporting element 2b.

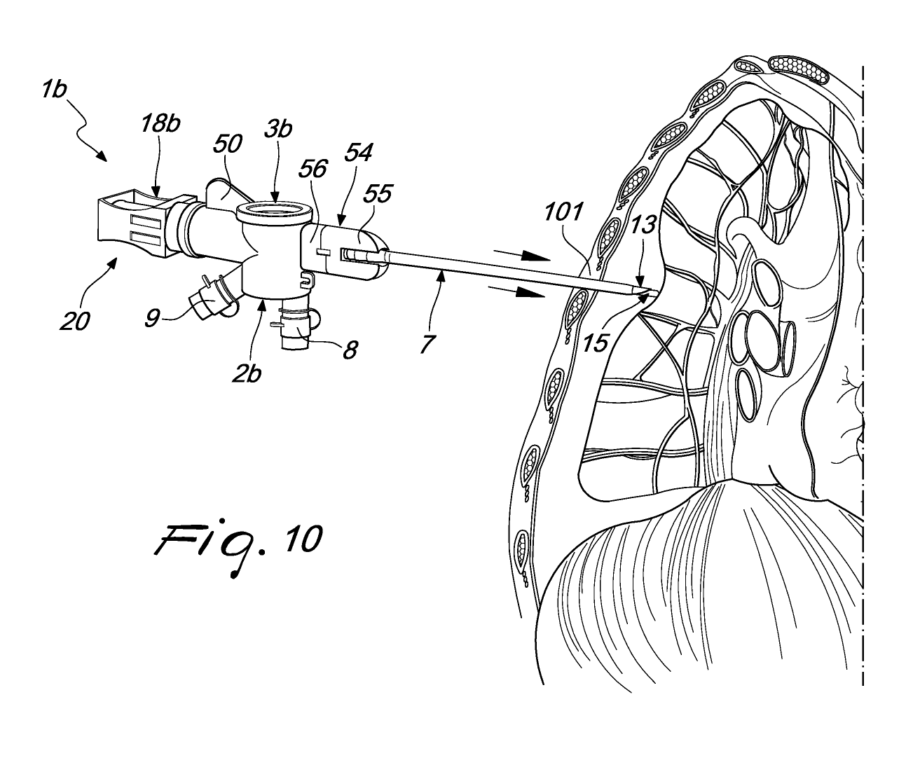

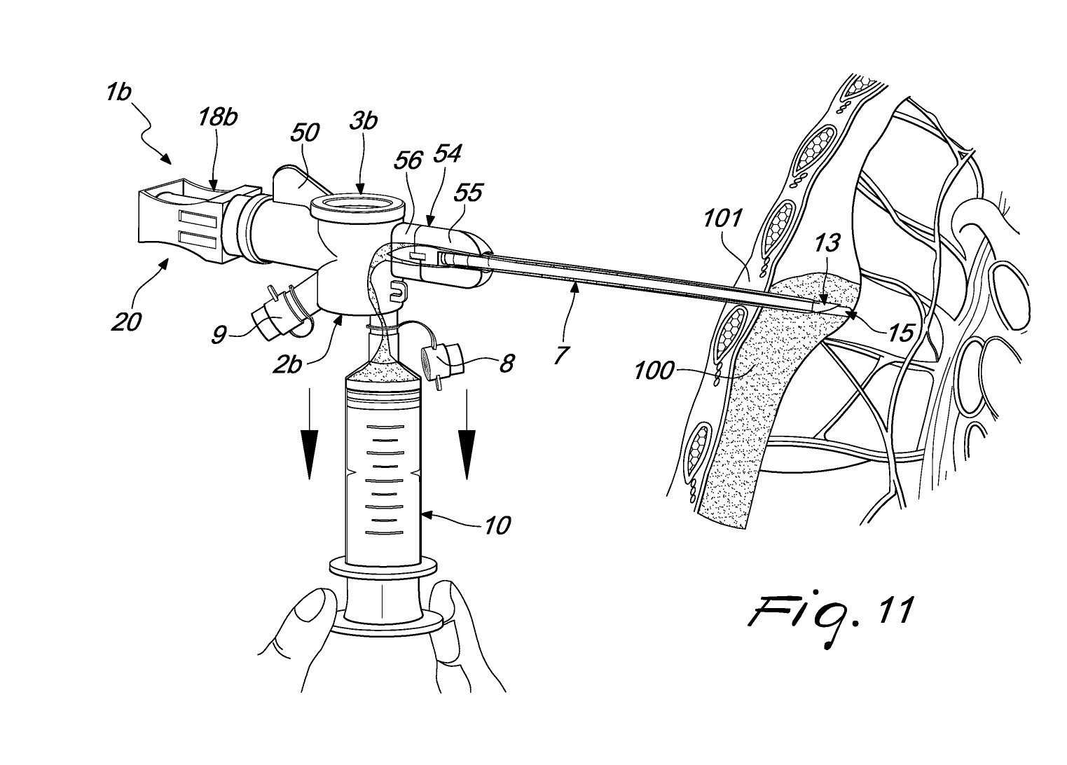

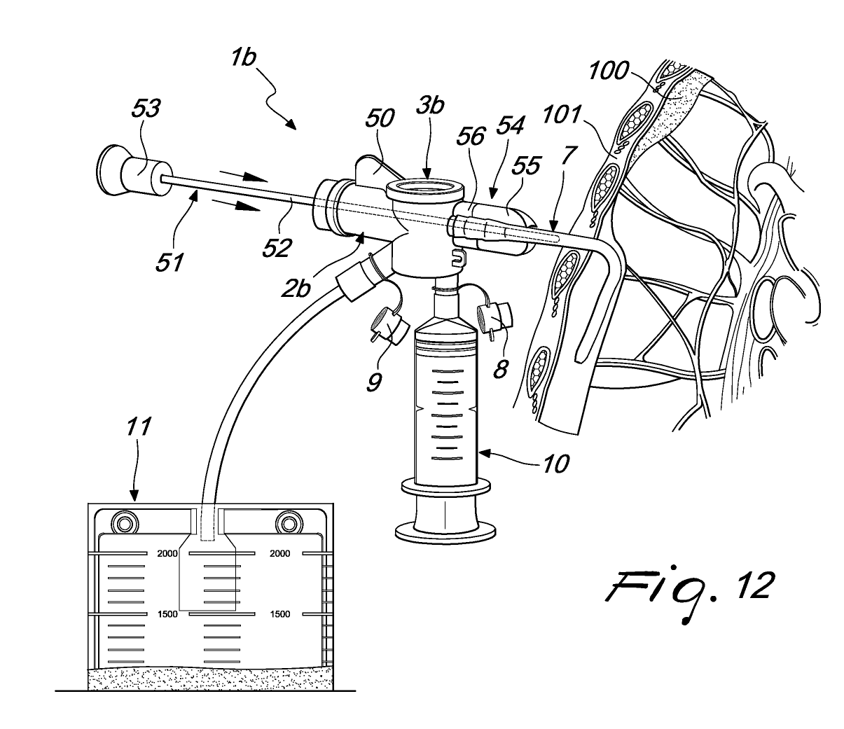

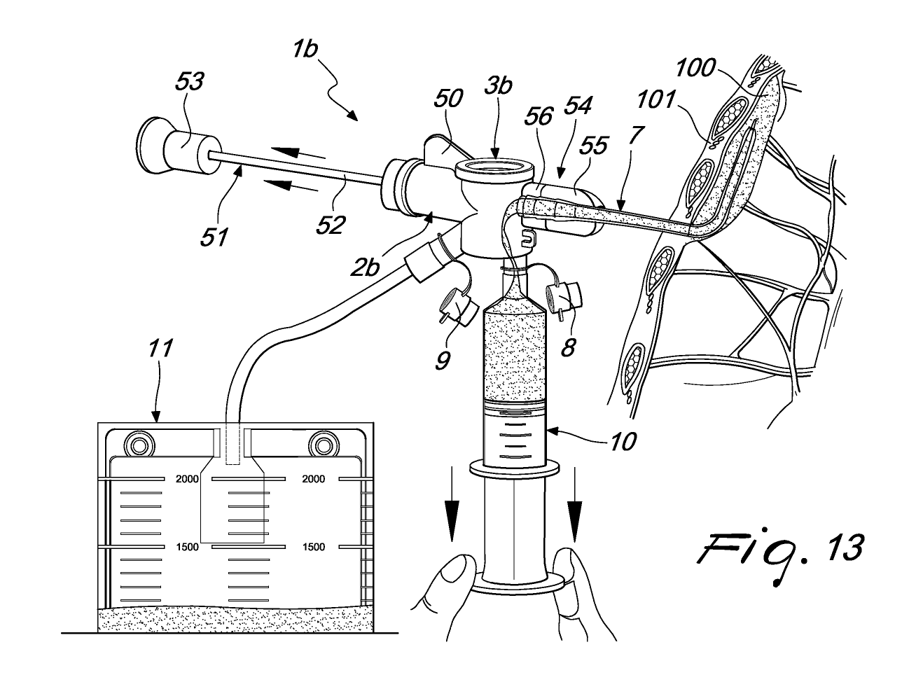

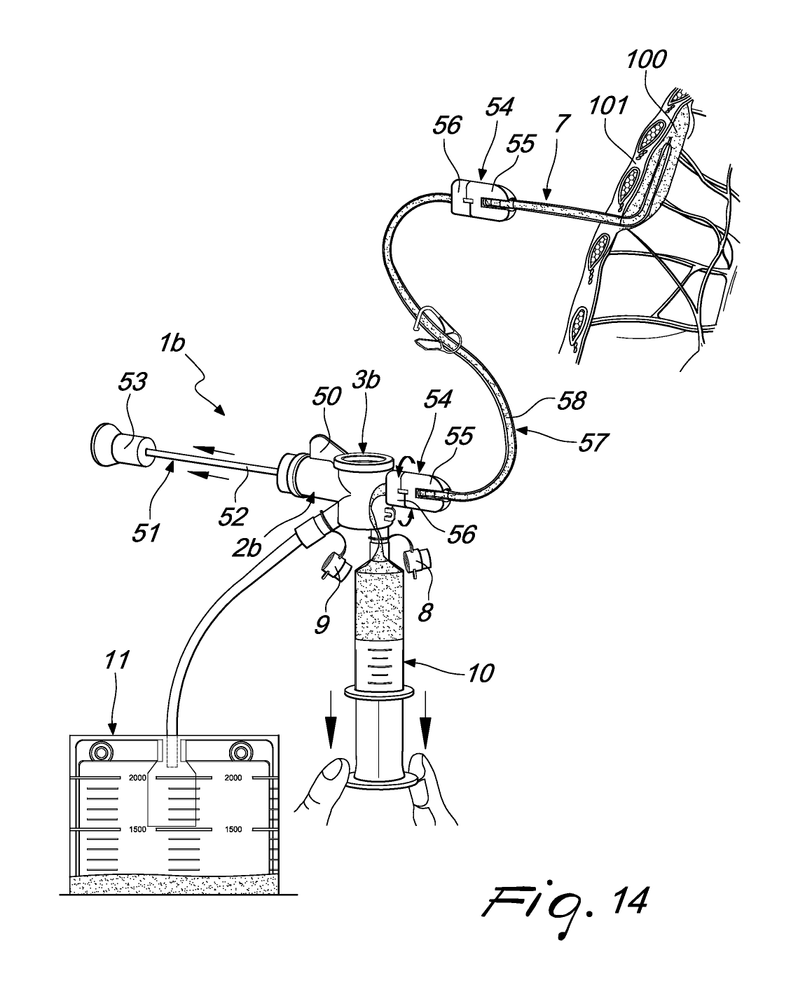

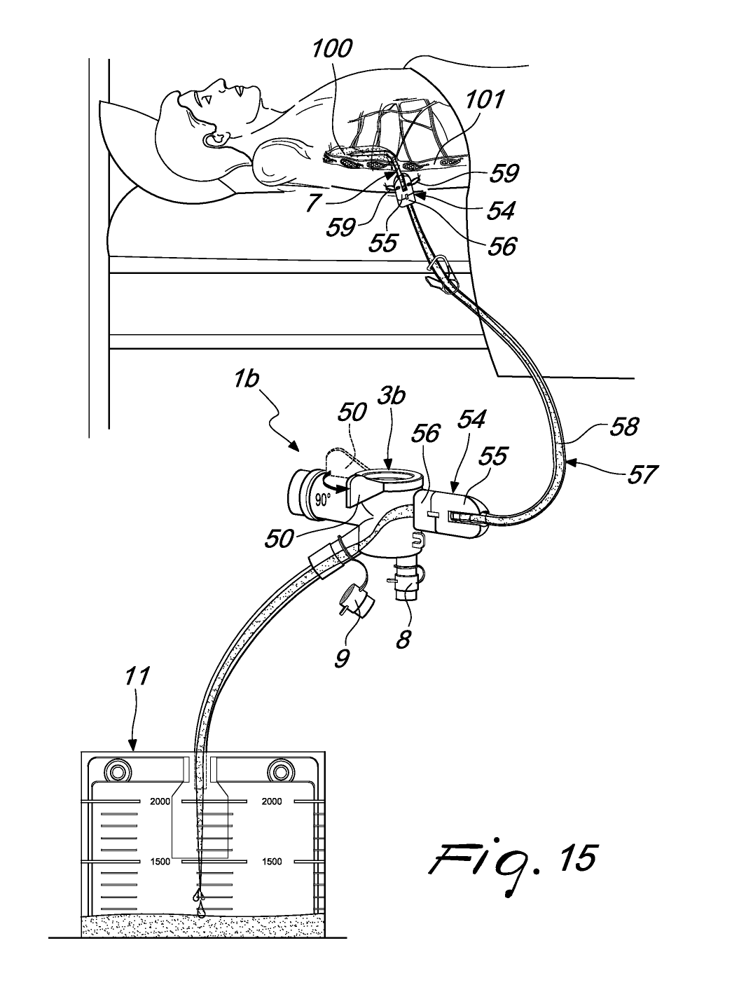

With reference to FIGS. 8 to 15, if there are pockets of fluid to be drained in different places in the patient, the cannula 7 would have to be inserted again with a different orientation from the previous one, so as to drain effectively all the fluid present in the pleural space or in other body cavities.

As will be described in more detail hereinafter, in order to allow the repositioning of the medical device 1b, as an alternative to the sharp-tipped needle 13 there is a spindle 51, which is composed substantially of a small round-tipped rod 52 and has, at the end that lies opposite the round tip, a grip body 53.

The spindle 51 can be used also in the first embodiment so as to allow the repositioning of the medical device 1a.

In order to further improve the comfort of the patient if the medical device 1b must remain inserted in place for a prolonged time, it is possible to space the supporting element 2b of the medical device 1b from the exit point of the catheter, in such a position as to not cause discomfort to the patient or damage to the skin, by means of one or more connecting elements 54.

More precisely, the connecting elements 54 comprise a male element 55 and a female element 56, which can be mutually associated by means of a coupling of the bayonet type and can be fixed respectively to the cannula 7 and to the opening 4b of the supporting element 2b.

As will be described better hereinafter, extension elements 57 can be provided which are constituted by a small tube 58 that has, at its ends, the elements 55 and 56 so that it is interposed between the cannula 7 and the supporting element 2b.

For keeping the medical device 1b applied for a prolonged period of time, avoiding the accidental risk that the catheter might move from the original position or even be extracted eyelet elements 59 are provided on the supporting element 2b and on the connecting elements 54.

In this manner is possible to easily fix these parts of the medical device lb to the skin of the patient, thus avoiding possible displacements of the position of the catheter or even accidental extraction thereof.

Application and operation of the medical devices 1a and 1b for applying catheters, particularly for thoracentesis procedures, are described hereinafter.

By gripping the medical device 1a or 1b by the supporting element 2a or 2b, the medical staff assigned to the application of the medical device 1a or 1b arranges said device so that the round tip 16 is directed toward the part of the thorax on which insertion is to be performed.

This insertion, which occurs generally on the rear part of the thorax just below the tip of the scapula, approximately in the eighth intercostal space, occurs by pushing forcefully the entire medical device 1a or 1b against the thorax of the patient, for example, by pushing the container body 18a or 18b.

This pressure causes the round tip 16 to retract into the sharp-tipped needle 13, leaving the sharp part 29 of the sharp-tipped needle 13 free to pierce the skin and the muscular tissues of the patient until the pleural space of the patient is reached.

This arrival can be ascertained by checking whether the slider 21a or 21b is visible in the container body 18a or 18b.

In the medical device the slider is in fact clearly visible before use. When instead the sharp-tipped needle 13 penetrates “hard” tissues, the slider 21a or 21b disappears behind the container body 18a or 18b. As soon as the sharp part 29 of the sharp-tipped needle 13 enters the pleural space, the round tip 16, since it no longer has a force which contrasts and opposes the force applied by the elastic means 17, exits again and with it the slider 21a or 21b returns to its visible position, so as to allow the medical staff to understand that piercing has been completed.

With such a solution, i.e., with the round tip 16 that exits again from the sharp-tipped needle 13 as soon as a “soft” part is reached, one avoids damaging tissues such as, for example, pulmonary tissue with the sharp portion 29 of the sharp-tipped needle 13.

Once the sharp-tipped needle 13, which carries the cannula 7 with it as well, has been inserted, it is possible to connect a syringe 10 and a collection bag 11 to the respective openings 5a and 6a or 5b and 6b.

This connection can also be performed before piercing of the patient's thorax.

In any case, by means of the groove or grooves 12 it is possible to check whether the tip of the cannula 7 leads into the pleural space as desired.

In fact, the biological liquid 100 to be drained reaches, by way of the grooves 12, the first opening 4a or 4b and from there, by way of the ducts formed by the selector 3a or 3b arranged in the first functional position, it arrives, through the one-way valve 30a or 30b, at the opening 5a or 5b to which a syringe is connected by means of which it is possible to check the flow of the drained biological liquid 100.

If the biological liquid 100 does not flow as described, the medical device 1a or 1b is oriented until the cannula 7 draws the biological liquid 100 correctly.

Once it has been ascertained that the piercing operation has been performed correctly, the sharp-tipped needle 13 is extracted from the medical device 1 by removing it and all the components connected thereto.

During this removal, there are no unwanted leaks because, as already mentioned, the first valve 27 and the membrane-type sealing gasket 28 are arranged at a distance from each other that is greater than the sum of the length of the sharp portion 29 of the sharp-tipped needle 13 and the stroke of the round tip 16 of the round-tipped element 15.

Once the sharp-tipped needle 13 has been extracted, leaving the selector 3a or 3b in its first functional position, the cannula 7 is connected to the inside of the selector 3a or 3b, but the presence of the one-way valve 30a or 30b and the valve 27 makes it possible to prevent the flow of air from the environment back toward the pleural space of the patient, allowing staff to operate in complete safety.

In order to drain the biological liquid 100, it is possible to connect the syringe to the opening 5a or 5b and a collection bag to the opening 6a or 6b.

When the piston of the syringe is retracted, the one-way valve 30a or 30b allows the liquid to pass and be collected in the syringe proper. When the piston of the syringe is pushed into its initial position, the one-way valve 30a or 30b prevents the biological liquid from flowing back toward the patient and thus such liquid is conveyed to the opening 6a or 6b and collected in the bag. The aspiration and collection operation, by acting on the piston of the syringe 10, can be performed several times without the need to act on the position of the selector, thus simplifying the execution of the procedure.

By rotating the selector 3a or 3b to its second functional position, the opening 14a or 14b is isolated, leaving in direct connection the other openings 4a and 6a or 4b and 6b while the opening 5a or 5b is isolated by the one-way valve 30a or 30b. In this manner, the biological liquid 100 can be collected directly by passing through the cannula 7, through the ducts 25, 24 and 26 toward the opening 6a or 6b, avoiding passage through the one-way valve 30a and 30b. In this manner, load losses caused by the one-way valve 30a or 30b are reduced, facilitating the outflow and collection of the biological liquid 100 by gravity.

If the level of the biological liquid 100 to be drained inside the pleural space drops below the drawing end of the cannula 7, it is possible by repositioning the control lever 22a or 22b in its first functional position to reinsert the sharp-tipped needle 13 in the cannula 7 so as to reorient it in the pleural space, so as to draw the remaining portion of the biological liquid 100 to be drained and repeat the procedure just described without however disconnecting the cannula 7 from the patient's body. As an alternative, for positioning of the medical device 1a or 1b, the spindle 51 can, at any time, be inserted from the opening 14a or 14b, performing simultaneously the partial extraction of the catheter.

With the spindle 51 inserted, but without complete extraction of the catheter, such catheter can be oriented in a different position, affecting the pocket where the fluids to be drained are located.

At this point, the catheter can be inserted completely and the spindle 51 can be extracted from the supporting element 2a or 2b.

The operation is entirely similar to the one performed during initial positioning, and therefore the physician does not have to learn different maneuvers, to the full benefit of ease of use of the medical device 1a or 1b.

Moreover, the use of a spindle 51 makes the procedure completely safe, avoiding any damage to pulmonary tissue.

Finally, the impossibility of connecting the thorax to the outside environment no matter what maneuver the physician performs makes it possible to change the position of the catheter whenever necessary, without limitations of a technical type to the number of repositionings.

Often catheter access, in thoracentesis procedures, is performed from the back, and therefore when the catheter is inserted completely the body is in such a position as to make it very difficult for the patient to stay in the supine position.

Thanks to the presence of extension elements 57 it is possible to move the fixing of the supporting element 2b with respect to the exit point of the catheter.

If it is necessary to fix the supporting element 2b of the medical device 1b in another position than the exit point of the catheter, it is possible to disconnect the cannula 7 and the supporting element 2b in order to insert one or more extension elements 57, mutually spacing the parts.

The bayonet couplings of the elements 55 and 56, of the male and female type, make it possible to perform the operation simply and rapidly and leave unchanged the properties of the medical device 1b of being connectable to different gravity- or suction-based collection systems.

Once the unit has been assembled, the end that lies proximate to the end portion of the cannula 7, i.e., where such cannula exits from the body of the patient, can be fixed to the skin by means of ordinary suture stitches. The other end of the extension line, i.e., the one connected to the supporting element 2b, can be in turn fixed in such a position as to allow the patient to assume different positions comfortably without feeling pain or discomfort and at the same time allow access and connection of the collection devices by medical staff.

The importance of the insertion of the extension elements 57 lies in the consequences of prolonged contact of the supporting element 2b of the medical device 1b with the skin of the patient.

In fact, if clinical conditions (e.g., prolonged loss, difficulties in drainage, problems due to accidental traumas, high density of the fluids to be drained . . . ) occur, the physician is forced to keep the medical device 1b in place for a prolonged period of time, even for several days.

The application of the extension elements 57 makes it possible to position the supporting element 2b of the medical device 1b in a position that is spaced from the exit point of the catheter, so that the patient is allowed to move freely and to assume any comfortable position.

The absence of contact between the supporting element 2b and the skin of the patient prevents the problems of pain and hematoma leading to necrosis, reducing complications and the infective risk.

The placement of the supporting element 2b in an accessible position allows medical and nursing staff to perform all necessary operations quickly and independently of the cooperative capacity of the patient.

In particular, the replacement of the collection system, the execution of diagnostic aspirations with the syringe, the execution of lavages, etcetera . . . are the operations most frequently required for correct management of the drainage.

The presence of said eyelet elements 59 both on the supporting element 2b and on the elements 55 and 56 allows to fix both parts in different positions if the extension line is used. By doing so, the supporting element 2b can be arranged where it does not cause inconvenience to the patient.

Moreover, besides the described thoracentesis and thoracic drainage procedures, the medical device 1a and 1b is suitable also for pericardiocentesis procedures, where the sharp-tipped needle 13 is inserted in the thorax and in the pericardiac membrane where the heart is contained.

All this is done to remove blood accumulations and avoid cardiac tamponade, i.e., cardiac arrest caused by the pressure of the accumulated fluids.

The structure of the medical devices la and lb is such that they can be used effectively in paracentesis procedures, where the liquids to be drained are located in the abdominal space below the diaphragm.

In practice it has been found that the medical device for applying catheters, particularly for thoracentesis procedures, according to the present invention, makes it possible to perform thoracentesis procedures without causing damage to the soft tissues of the patient during the application of the catheter, giving constant feedback on the compactness of the tissue that the sharp-tipped needle is penetrating and most of all allowing the repositioning of the catheter inside the pleural space without replacing it and without detaching any syringes or collection bags from the medical device itself

Another advantage of the medical device according to the present invention is that it is easy to use and structurally simple so as to have low manufacturing costs.

Another advantage of the medical device according to the present invention is that it is possible to perform aspiration of the fluids to be drained, by means of a syringe, without having to switch the selector valve at each stroke of the piston of the syringe, greatly simplifying this operation.

The invention is further advantageous by way of the absence of a one-way valve in the rear grip of the sharp-tipped needle, despite maintaining the possibility to perform an exploratory aspiration in order to determine the correct positioning of the needle.

Said valve, in fact, is integrated in the body of the device. Therefore the device is more compact, simpler and easier to handle, entirely to the advantage of the physician who uses it.

The medical device for applying catheters, particularly for thoracentesis procedures, according to the present invention is susceptible of numerous modifications and variations.

For example, the slider, which is accommodated in the container body, contrary to what has been stated so far, can be movable from a non-visible position to a visible one, respectively, in which the round tip protrudes at least partially from the sharp-tipped needle (non-visible position of the slider) and in which the round tip is completely retracted into the sharp-tipped needle (visible position of the slider).

All the details may further be replaced with other technically equivalent elements.

In practice, the materials used, as long as they are compatible with the specific use, as well as the contingent shapes and dimensions, may be any according to requirements and to the state of the art.

1-15. (canceled)

16. A medical device for applying catheters, particularly for thoracentesis procedures, comprising a hollow supporting element which can be gripped ergonomically and accommodates a selector which is operated manually and is adapted to connect at least three openings defined in said supporting element, the first one of said openings being functionally associated with a cannula that is adapted to be inserted in the body of a patient for the drainage of biological liquid or for the injection of drugs, the second opening and the third opening being able to be functionally associated respectively with two closure elements or with at least one of a syringe and a collection bag, a sharp-tipped needle being slidingly insertable in said cannula and having such a length as to pass through at least all of said cannula and protrude from said cannula to pierce the biological tissue that lies in the vicinity of said biological liquid, further comprising a fourth opening defined in said supporting element on the side opposite said first opening for extraction or insertion of said sharp-tipped needle respectively from and into said cannula.

17. The medical device according to claim 16, further comprising a round-tipped element, which is inserted slidingly in said sharp-tipped needle so that a round tip thereof protrudes from said sharp-tipped needle on the side opposite said supporting element, said round tip being retractable into said sharp-tipped needle in opposition to the action of elastic means associated with said round-tipped element.

18. The medical device according to claim 17, wherein said elastic means comprise a helical spring, which is accommodated in a container body that is jointly connected to said sharp-tipped needle and is interposed between one end of said round-tipped element and an internal abutment surface of said container body, said container body being engageable with said supporting element at said fourth opening.

19. The medical device according to claim 17, further comprising means for visualizing the retraction of said round tip into said sharp-tipped needle.

20. The medical device according to claim 19, wherein said visualization means comprise a slider, which is associated with said round-tipped element on the opposite side with respect to said round tip, is accommodated in said container body and can move from a visible position, in which said round tip protrudes at least partially from said sharp-tipped needle, to a non-visible position, in which said round tip is completely retracted into said sharp-tipped needle, said container body being at least partially transparent for viewing said slider in said visible position.

21. The medical device according to claim 16, further comprising manually actuated control means for said selector, comprising a control lever, which can move from a first functional position to a second functional position, said four openings being mutually connected when said control lever is arranged in said first functional position and said first opening, said second opening and said third opening being mutually connected when said control lever is arranged in said second functional position.

22. The medical device according to claim 21, wherein said manually actuated control means comprise a flap which is rigidly coupled to said control lever and protrudes along a direction that is substantially parallel to the axis of rotation of said selector inserted in said supporting element.

23. The medical device according to claim 18, further comprising a first valve of the one-way type, which is normally closed, is accommodated in said supporting element, is interposed between said selector and said fourth opening, and is provided with a membrane-type sealing gasket that can be engaged by abutment against said container body, said first valve having a central slit which is elastically deformable for the passage of said sharp-tipped needle in both directions.

24. The medical device according to claim 23, wherein said first valve and said membrane-type sealing gasket are arranged with respect to each other at a distance that is larger than the sum of the length of the sharp part of said sharp-tipped needle and the stroke of said round tip of said round-tipped element to prevent unwanted leaks of said biological liquid to be drained during the operations for inserting and extracting said sharp-tipped needle respectively in and from said cannula.

25. The medical device according to claim 16, further comprising at least one groove that is formed on the internal surface of said cannula and runs longitudinally with respect to said cannula for the connection of said first opening to said biological liquid to be drained.

26. The medical device according to claim 23, further comprising a second valve of the one-way type, which is accommodated in said supporting element and is interposed between said selector and said second opening.

27. The medical device according to claim 16, further comprising a spindle having a rod, which can be slidingly inserted in said cannula as a substitution for said sharp-tipped needle and has such a length as to pass through at least all of said cannula and protrude from said cannula for repositioning of said cannula in the patient.

28. The medical device according to claim 16, further comprising at least one connecting element provided by a male element and a female element, which can be mutually associated and fixed, respectively, to said supporting element at said first opening and to said cannula.

29. The medical device according to claim 28, further comprising at least one extension element provided by a tube provided, at its ends, with said male element and said female element and can be interposed between said male element of said supporting element and said female element of said cannula.

30. The medical device according to claim 28, further comprising eyelet elements, which are formed in said supporting element and in said at least one connecting element.
